(19) United States
(12) Patent Application Publication
(10) Pub. No.: US 2012/0203285 A1
(43) Pub. Date: Aug. 9, 2012

(54) DEVICE FOR SYNTHESIS OF BONE FRACTURES

(75) Inventors: Roberto Rotini, Bologna (IT); Enrico Guerra, San Lazzaro di Savena (IT)

(73) Assignee: Hit Medica SPA, Serravalle (SM)

(21) Appl. No.: 13/500,298

(22) PCT Filed: Oct. 5, 2010
(86) PCT No.: PCT/EP2010/064781

§ 371 Date: Apr. 4, 2012

(30) Foreign Application Priority Data
Oct. 5, 2009 (SM) SM-A-200900081

(51) Int. Cl. A61B 17/80

(52) U.S. Cl. 606/286

(57) ABSTRACT

Device for synthesis of bone fractures, comprising a plate and screws for locking said plate. The plate is entirely made from biocompatible and radiolucent plastic material suitable to permit a radiographic control and an easy explantation of the plate and is provided with through holes for the screws fixing the plate on the bone. The screws comprise a threaded shank having an external diameter less than the diameter of said holes and a head having a thread with an external diameter greater than the diameter of said holes so as to permit insertion of the screws according to a plurality of directions and inclination with respect to the axis of the holes in the plate, said head being suitable to engage said holes by self-threading.

### BACKGROUND OF THE INVENTION

1. Field of the Invention

The present invention relates to a device for synthesis of bone fractures.

2. Description of Related Art

For synthesis of bone fractures the use of special metal plates is known which are properly shaped to be locked on the outer side of the bone by means of screws. For example, for synthesis of trochanter or femur neck fractures plates, are used which are placed on the fracture edges and fixed by means of screws which are driven with proper inclination in order to penetrate the bone parts which appear to be solid and less injured and therefore able to assure a firm reduction of the fracture.

However the plates generally used present some locking difficulties owing to the fact that the plates do not always permit that the screws are steadily inserted into the fractured segments of the bone with exact and sufficient inclination. In order to permit that the screws assume a greater inclination, it has been suggested to insert an auxiliary bushing or washer between the plate and the screw head having a hemispherical seat to accommodate the complementary head of the screws so as to create an articulation allowing the screws to assume the required inclination. On the other hand the interposition of bushings involves evident economic increase and their use requires an appreciable rise of the plate dimension to get sufficient space for the creation of a hemispherical seat suitable to lodge the screw head.

### BRIEF SUMMARY OF THE INVENTION

The aim of the present invention is therefore to achieve a device capable to overcome the drawbacks of the known devices and to be economically profitable compared with the traditional ones.

This aim is achieved with a device for synthesis of bone fractures, comprising a plate and screws for locking said plate. Said plate is made from biocompatible and radiolucent plastic material suitable to permit a radiographic control and an easy explantation of the plate and is provided with through holes for the screws fixing the plate on the bone. Said screws comprise a threaded shank having an external diameter less than the diameter of said holes and a head having a thread with an external diameter greater than the diameter of said holes so as to permit insertion of the screws according to a plurality of directions and inclination with respect to the axis of the holes in the plate, said head being suitable to engage said holes by self-threading.

According to some embodiments of the present invention, the holes of the plate are substantially cylindrical and have a diameter such as to permit angular selection of the screw shank relative to the plate. Moreover, in some embodiments, the head is substantially cylindrical and has a helical thread with spiral rings having an external diameter greater than the diameter of the holes so that, when the head is screwed in the holes, the spiral rings engage the hole wall by self-threading so as to lock the screw in the selected angulation.

### BRIEF DESCRIPTION OF THE SEVERAL VIEWS OF THE DRAWINGS

Further characteristics and advantages of the present invention will become more apparent from the following description of a preferred but not exclusive embodiment, illustrated by way of example in the accompanying drawings wherein:

FIG. 1 shows a perspective view of a plate according to the invention;

FIG. 2 is an enlarged view of a portion of the plate of FIG. 1;

FIG. 3 is a view of a screw partially screwed through the plate;

FIG. 4 is a view of a screw entirely screwed through the plate of FIG. 1.

### DETAILED DESCRIPTION OF THE INVENTION

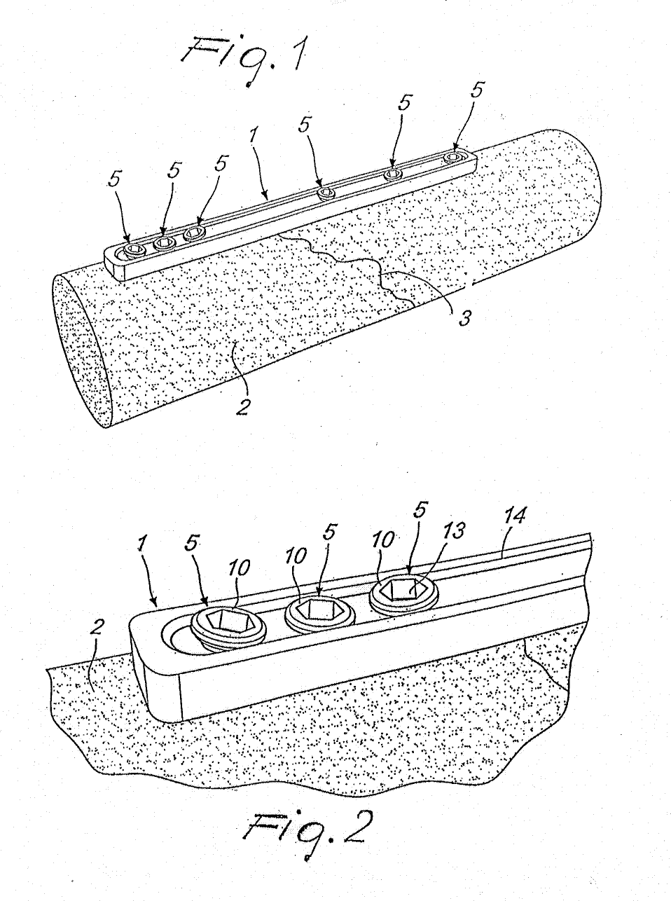

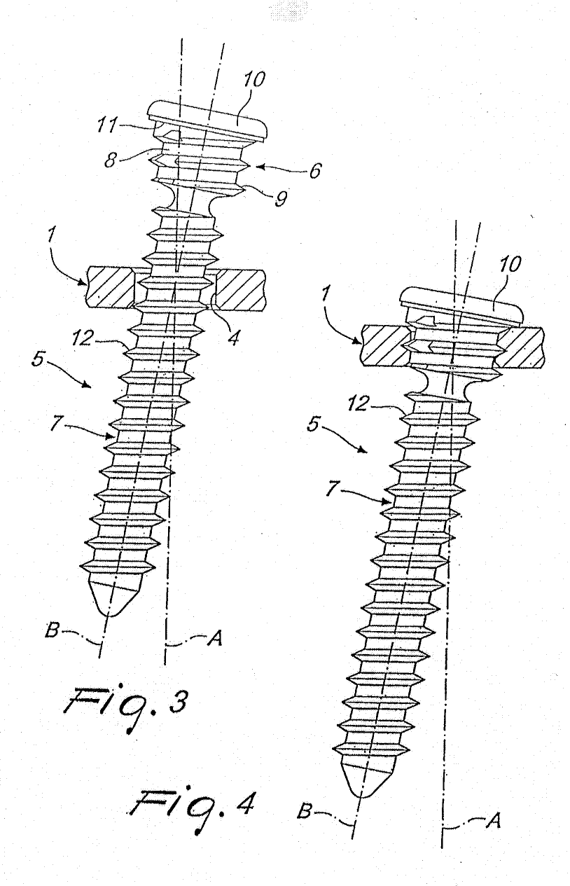

In FIGS. 1 and 2 the reference 1 designates a plate according to the invention comprising a bar entirely made from a biocompatible plastic material POLYETHRETHERKETONE, well known by name of PEEK and suitable to facilitate the explantation of the plate. Preferably this biocompatible plastic material is radiolucent to make the radiocontrol easy and advantageously strengthened by means of carbon fibres.

The shape of the plate may be any according to the shape of the bone near to the fracture to be reduced. In FIG. 1, references 2 and 3 indicate the bone and the fracture edges respectively. In plate 1 a plurality of through holes is formed (see FIG. 4) the number and disposition of which along the plate depend from the design of the plate and from the anatomical zones in which the use is intended. The holes 4 are preferably cylindrical and their diameter depends from the size of screws 5 which are driven through the holes 4 to lock the plate 1 on the bone 2 and to join the bone portions that compose the fracture 3.

The screws 5 comprise a head 6 and a shank 7. The head 6 comprises a core 8, around which a helical thread 9 extends helically having one or two starts according to the screw type, whereby the spiral rings of the threads have a substantially triangular or orthopaedic section and an external diameter which is greater than the diameter of the hole 4. In some embodiments, the head 6 is cylindrical in shape. In some particular embodiments, the core 8 is cylindrical and has a diameter smaller than that of the hole 4 so that, when the head 6 is screwed in the hole 4, the thread 9 engages the wall of the hole 4. Likewise the core 8 may present a conical shape.

The head 6 at the end opposite to the shank 7, is provided with a collar 10 having a diameter greater than the external diameter of the thread 9, which collar 10 acts as arrest on the upper face of the plate 1 when the head 6 is completely threaded in the hole 4 as it will appear in the following. Advantageously, in order to render the screw 5 more secure against unscrewing once it is tightened, the lower face of the collar 10 comprises a indentation 11 which engages the plate 1 thus preventing unscrewing of the screw 5 in case of bone failure.

The shank 7 comprises a thread 12, for example having spiral rings with substantial triangular or usual in orthopaedic threads and an external diameter smaller than that of the core 8 and therefore such as to render the shank 7 loose when driven through the hole 4. The difference between the diameter of the hole 4 of the plate 1 and the external diameter of the thread of the shank 7 is preferably such as to permit the latter to reach an inclination of at least 15° with respect to the axis A of the hole 6.

The structure of the screws 5 is completed by a recess 13 formed in the head 6 (see FIG. 4) and having a polygonal shape to permit engagement of an alien spanner by means of which the user may carry out screwing of the screws.

From the above description it will appear evident that the plate 1 may be positioned with high simplicity. Indeed after the plate 1 has been positioned on the bone 2, the screws 5 are applied and the axis B may be arranged with respect to axis A of the holes, according to the inclination necessary to drive the screws into the various areas of the bone 2 which are in condition to give a sufficiently firm grip able to consent reduction of the fracture 3. In a first phase when the plate 1 has been positioned on the bone 2, by means of an alien spanner engaging the recess 13, the screw 5 is screwed until the thread 9 of the head 6 abuts the plate 1. At this moment, a further screwing of the screw 5 causes abutment of the plate 1 on the bone 2 owing to the pressure of the first spiral ring of the thread 9 of the head 6 on the plate 1. When the pressure exerted by the thread 9 on the plate 1 is over a certain value, forced penetration of the thread 9 into the hole 4 begins and the collar 10, acting on the plate 1, causes the definitive tightening on the bone 2.

It will be observed that while locking of the plate 1 on the bone 2 is resulting from a first traction effect exerted by the thread 12 of the shank 7, screwing of the thread 9 gives as result to set at zero the clearance of the screw 5 in the hole 4 and therefore the even if very small displacement that the plate 1 could do and compromise the firmness of the plate 1 and of the fracture 3.

In fact, in order to permit the inclination of the, screws 5 according to the necessity required from the appearance of the fracture 3, it would be necessary to provide increase of the hole dimension. However the increase would cause excessive clearance which will have as consequence a locking inadequate and suitable to induce displacement of the plate 1.

On the contrary the present invention suggests a solution with the substantial advantage that the head 6, owing to a thread 9 having a greater diameter, after being forced into the hole 4, permits to nullify the clearance of the increased holes 4 and consequently to firmly lock the plate 1. Advantageously, in order to create a compression effect between plate 1 and bone 2, the threads 9, 12 have a small pitch difference.

Moreover, with the present invention the selection of angulation and locking of the screws is not limited as in the prior art. In fact, the plastic material, e.g. PEEK, of the plate permits the threads of the screws to penetrate by self-drilling the inner wall of the hole also in case of relevant angulation with respect to the plane thus achieving solid anchoring of the screws to the plate.

Thus the described device achieves the intended aims. The use of radiolucent plastic material, unlike to a radio-opaque material (stainless steel, titanium alloy of common use) permits a more efficacious radiographic control of the fracture both in operating field, including the possibility to improve the implantation of the plate, and during periodical post operative controls in order to verify the progress of the recovery of the fracture. Another very important aspect of the invention is the possibility to easily explant the plate 1, contrary to the difficulties found in the explantation of metal plates having threaded holes. In fact in these plates the head of the screws engages the threaded hole of the plate and the unscrewing of the screws during explantation is laborious because of the threads which get stuck between the screw head and the plate.

Numerous modifications and variations are possible in the practical embodiment of the invention. Advantageously the plate 1 is provided with a perimetric stiffening rib 14 (see FIGS. 1, 2). Instead of one rib it is possible to provide few ribs arranged so as to stiff the zones of the plate which are subjected to the greatest efforts.

1. A device for synthesis of bone fractures. comprising a plate and screws for locking said plate, wherein said plate is entirely made from biocompatible and radiolucent plastic material suitable to permit a radiographic control and an easy explantation of the plate and is provided with through holes for the screws fixing the plate on the bone and wherein said screws comprise a threaded shank having an external diameter less than the diameter of said holes and a head having a thread with an external diameter greater than the diameter of said holes so as to permit insertion of the screws according to a plurality of directions and inclination with respect to the axis of the holes in the plate, said head being suitable to engage said holes by self-threading, wherein said holes of the plate are substantially cylindrical and have a diameter such as to permit angular selection of the screw shank relative to the plate, said head being substantially cylindrical and having a helical thread with spiral rings having an external diameter greater than the diameter of the holes so that, when the head is screwed in the holes, the spiral rings engage the hole wall by self-threading so as to lock the screw in the selected angulation.

2. The device according to claim 1, wherein said radiolucent material is suitable to permit a radiographic control during reduction of the fracture,

3. The device according to claim 1, wherein said head, at the end opposite to the shank, is provided with a collar having a diameter greater than the external diameter of the threading of said head and suitable to act as abutment on the upper face of the plate when the head is completely screwed in said hole.

4. The device according to claim 3, wherein said collar is provided with an indentation on the face facing said threading.

5. The device according to claim 1, wherein said biocompatible and radiolucent plastic material is POLYETHERETHER-KETONE known on the market as PEEK.

6. The device according to claim 5, wherein said biocompatible and radiolucent plastic material is POLYETHERETHER-KETONE known on the market as PEEK and stiffened with carbon fibres.

7. The device according to claim 1, wherein said biocompatible and radiolucent plastic material is suitable to aid explantation of the head of the screws from the holes of said plate.

8. The device according to claim 1, wherein said plate is provided with stiffening ribs.

9. The device according to claim 8, wherein said plate is provided with a perimetric stiffening rib.

10. (canceled)
